(19) United States
(12) Patent Application Publication
(10) Pub. No.: US 2019/0231893 A1
(43) Pub. Date: Aug. 1, 2019

(54) METHODS OF TREATING CANCER

(71) Applicant: AbbVie Inc., North Chicago, IL (US)

(72) Inventors: Peter Ansell, Grayslake, IL (US); Lei He, Northbrook, IL (US); Xin Lu, Libertyville, IL (US); Lisa Roberts-Rapp, Gurnee, IL (US); Zheng Zha, Buffalo Grove, IL (US)

(21) Appl. No.: 16/195,384

(22) Filed: Nov. 19, 2018

Related U.S. Application Data
(60) Provisional application No. 62/588,287, filed on Nov. 17, 2017.

(51) Int. Cl. A61K 47/68; A61P 35/00; C12Q 1/6886; C07K 16/22

(52) U.S. Cl. CPC A61K 47/6803; A61P 35/00; A61K 47/6865; C07K 16/22; C12Q 1/6886

(57) ABSTRACT

The present disclosure provides a method for treating a patient having cancer, such as glioblastoma. The method may comprise obtaining a biological sample from the patient, and detecting a mutation in a microtubule-related gene is present in the biological sample. The method further comprises administering an effective amount of a pharmaceutical composition comprising a tubulin polymerization inhibitor, such as an auristatin, to the patient.

### CROSS REFERENCE TO RELATED APPLICATIONS

This application claims benefit to U.S. provisional application Ser. No. 62/588,287 filed Nov. 17, 2017 under 35 U.S.C. § 119(e). The entire teachings of the referenced application are incorporated herein by reference.

### BACKGROUND

Overexpression of EGFR has been reported in numerous human malignant conditions, including cancers of the bladder, brain, head and neck, pancreas, lung, breast, ovary, colon, prostate, and kidney. (Atalay et al., Ann. Oncology 14:1346-1363 (2003); Herbst and Shin, Cancer 94:1593-1611 (2002); and Modjtahedi et al., Br. J. Cancer 73:228-235 (1996)). In many of these conditions, the overexpression of EGFR correlates or is associated with poor prognosis of the patients. (Herbst and Shin, Cancer 94:1593-1611 (2002); and Modjtahedi et al., Br. J. Cancer 73:228-235 (1996)). EGFR is also expressed in the cells of normal tissues, particularly the epithelial tissues of the skin, liver, and gastrointestinal tract, although at generally lower levels than in malignant cells (Herbst and Shin, Cancer 94:1593-1611 (2002)).

Glioblastoma multiforme (GBM) is the most common and most aggressive type of primary brain tumor in adults. Patients diagnosed with glioblastoma have a poor prognosis, with a median overall survival ranging from 14-16 months, and a 24-month survival of approximately 30%. Stupp R et al., Effects of radiotherapy with concomitant and adjuvant temozolomide versus radiotherapy alone on survival in glioblastoma in a randomized phase III study: 5-year analysis of the EORTC-NCIC trial. Lancet Oncol., 2009; 10(5):459-466. Treatment for glioblastoma remains challenging. Omuro A and DeAngelis L. Glioblastoma and other malignant gliomas: A clinical review. JAMA. 2013; 310(18):1842-1850. Standard treatment is surgical resection, radiotherapy, and concomitant adjunctive chemotherapy. Relapse after initial therapy is common.

Amplifications of the EGFR gene (i.e., multiple copies of the EGFR gene), and the subsequent overexpression of EGFR protein, occur in approximately 40-60% of GBM patients. See Brennan C W, Verhaak R G, McKenna A, Campos B, Noushmehr H, Salama S R, et al. The somatic genomic landscape of glioblastoma. Cell 2013; 155(2):462-77; Yoshimoto K, Dang J, Zhu S, Nathanson D, Huang T, Dumont R, et al. Development of a real-time RT-PCR assay for detecting EGFRvIII in glioblastoma samples. Clin Cancer Res 2008; 14(2):488-93.

There remains a continuing need to develop methods of treatment for glioblastoma that effectively and selectively pair EGFR-targeting therapies with specific patients and patient populations, beyond merely EGFR amplification status.

### BRIEF SUMMARY

In embodiments, the present disclosure provides a method for treating cancer, such as glioblastoma, in a patient having a mutation in at least one gene in the γ-tubulin ring complex. In embodiments, the at least one gene is TUBGCP4 and/or TUBGCP6. In embodiments, the method comprises administering to the patient an effective amount of a therapy comprising a tubulin polymerization inhibitor. In embodiments, the tubulin polymerization inhibitor is an auristatin. In embodiments, the therapy is depatuxizumab mafodotin.

In embodiments, the present disclosure provides a method of treating cancer in an individual in need thereof, where the method comprises identifying a cancer cell from the patient as comprising a mutation in the γ-tubulin ring complex, and once the mutation has been identified, administering a treatment comprising a tubulin polymerization inhibitor to the patient. In embodiments, the at least one gene is TUBGCP4 and/or TUBGCP6. In embodiments, the tubulin polymerization inhibitor is an auristatin. In embodiments, the treatment is depatuxizumab mafodotin.

In embodiments, the present disclosure provides a method for treating glioblastoma in an individual in need thereof, where the individual has a mutation in a gene in the γ-tubulin ring complex selected from the group consisting of TUBGCP4 and TUBGCP6. In embodiments, the method comprises administering to the individual a therapeutically effective amount of depatuxizumab mafodotin. In embodiments, the individual has a mutation in TUBGCP4. In embodiments, the individual has a mutation in TUBGCP6.

### BRIEF DESCRIPTION OF THE DRAWINGS

FIG. 1 provides a waterfall plot illustrating the change in tumor size in patients with EGFR-amplified recurrent glioblastoma (rGBM) that were treated with either depatuxizumab mafodotin (denoted as “depatux-m”) alone or depatux-m in combination with Temozolomide (denoted as “TMZ”). As shown, although all responders exhibited EGFR amplification, not all patients with EGFR amplification had responded.

FIGS. 2A, 2B, and 2C provide a summary of the results of whole exome sequencing (WES) performed on 48 patient tumor samples to identify biomarkers associated with warhead sensitivity. The dataset included samples from 44 rGBM and 4 nGBM patients. Genes/pathways were identified that differentiated responders vs. non-responders and were selected based on highest significance as determined by fold change differentiation, P value, and false discovery rate. A trend was observed with combinatorial patterns of tubulin genes which were more prevalent in responders vs. non-responders. Some genes in the TUBA, TUBB, TUBG, and TLL gene families were mutated at higher levels across all response groups. The γ-tubulin ring complex, which is required for microtubule formation, includes TUBGCP4 and TUBGCP6. TUBGCP4 was mutated in responders (1/8) vs. non-responders (0/24). One patient with long-term SD had mutations in TUBGCP4 and TUBGCP6, both of which are in the same complex. The full results are shown in FIG. 2A (Responder), FIG. 2B (Stable Disease), and FIG. 2C (Non-responder).

FIG. 3 shows results from siRNA knockdown experiments of the differentially mutated γ-tubulin ring complex genes. As shown, siRNA knockdown of the differentially mutated genes impacted sensitivity to tubulin polymerization inhibitors (depatux-m, MMAE, and vincristine), but not to depolymerization inhibitors (paclitaxel).

FIG. 4 illustrates significant differences in gene expression (p<0.05) of the γ-tubulin ring complex genes were observed for TUBG1, TUBB2A, TUBB3, TTLL4, and TTLL7 between responders and non-responders. In FIG. 4, “SD” refers to stable disease, “R” refers to responder, “NR” refers to non-responder, and “FPKM” refers to Fragments Per Kilobase of Transcript per Million mapped reads.

### DETAILED DESCRIPTION

The present disclosure relates to the use of a therapy comprising tubulin polymerization inhibitors for the treatment of tumors having mutations of microtubule-related genes, in particular genes within the γ-tubulin ring complex. In embodiments, the tubulin polymerization inhibitor is an auristatin. In embodiments, the therapy is depatuxizumab mafodotin (“Depatux-m”, or “ABT-414”), an antibody-drug conjugate that preferentially binds cells with EGFR amplification. Depatux-m is composed of an EGFR IgG1 monoclonal antibody (depatuxizumab) conjugated to the tubulin inhibitor monomethyl auristatin F via a stable maleimidocaproyl link.

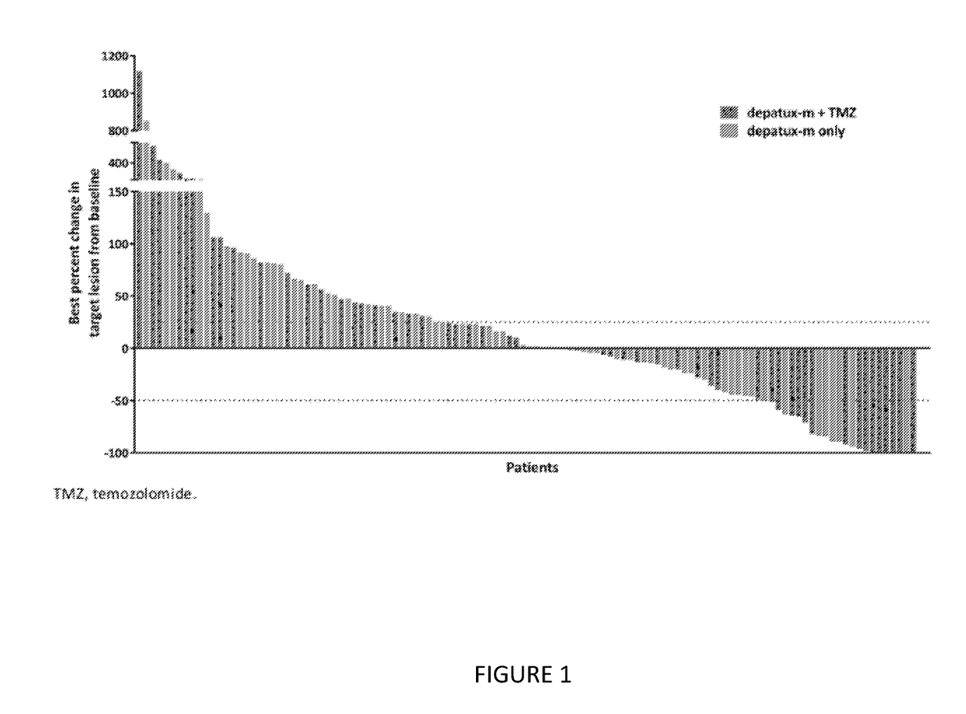

In a Phase 1 study (M12-356, NCT01800695) of depatuxizumab mafodotin in patients with newly diagnosed glioblastoma (nGBM) and recurrent glioblastoma (rGBM) with EGFR amplification, responses in patients with rGBM correlated with EGFR amplification in pre-treatment, archival tumor tissue. Gene amplification was determined by fluorescence in situ hybridization (FISH). Two probes were used: locus-specific EGFR probe and chromosome enumeration probe (CEP) 7. FIG. 1 illustrates change in tumor size in patients with EGFR-amplified rGBM. As shown in FIG. 1, while all responders exhibited EGFR amplification in the tumor, not all patients with EGFR amplification responded. That is to say, while all responders exhibited EGFR amplification, EGFR amplification alone was not necessarily sufficient for predicting clinical response for depatuxizumab mafodotin.

The present disclosure provides methods of identifying glioblastoma patients for treatment with therapies comprising a tubulin polymerization inhibitor, such as depatux-m, beyond merely EGFR amplification status. As discussed in more detail below, tumors having mutations in a microtubule-related gene, more particularly a mutation in a gene within the γ-tubulin ring complex, are sensitive to treatment with therapies comprising tubulin polymerization inhibitors, including depatux-m. Several proteins including γ-tubulin, TUBGCP4 and TUBGCP6 form the γ-tubulin ring complex (γ-TuRC), which is required for microtubule formation. In embodiments, the mutation in the γ-tubulin ring complex comprises a mutation in TUBGCP4 and/or TUBGCP6.

As used herein, the terms “treat,” “treating,” and “treatment” refer to a method of alleviating or abrogating a disease and/or attendant symptoms.

The term “patient” refers to a human or non-human subject who is being treated, monitored, or the like for a medical condition or disease. In embodiments, the patient is a human subject.

As used herein, the term “effective amount” or “therapeutically effective amount” refers to the amount of a drug (e.g., a tubulin polymerization inhibitor, an auristatin, an ADC such as depatuxizumab mafodotin) which is sufficient to reduce or ameliorate the severity and/or duration of a disorder, e.g., cancer, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of a drug may, for example, inhibit tumor growth (e.g., inhibit an increase in tumor volume), decrease tumor growth (e.g., decrease tumor volume), reduce the number of cancer cells, and/or relieve to some extent one or more of the symptoms associated with the cancer. The effective amount may, for example, improve disease free survival (DFS), improve overall survival (OS), or decrease likelihood of recurrence.

The term “administering” as used herein is meant to refer to the delivery of a substance (e.g., an anti-EGFR antibody-drug conjugate such as depatuxizumab mafodotin) to achieve a therapeutic objective (e.g., the treatment of an EGFR-associated disorder).

The term “auristatin”, as used herein, refers to a family of antimitotic agents. Auristatin derivatives are also included within the definition of “auristatin.” Examples of auristatins include, for example, auristatin E (AE), monomethylauristatin E (MMAE), monomethylauristatin F (MMAF), and synthetic analogs of dolastatin.

The term “tubulin polymerization inhibitor” refers to a compound which interacts with tubulin and inhibits tubulin polymerization and/or depolymerizes tubulin, and as a result, interferes with the physiological function of microtubules. In contrast, tubulin depolymerizing inhibitors, such as taxanes, stabilize tubulin polymers and/or inhibit tubulin depolymerization.

The term “positive EGFR status” refers to a cancer patient that has either a tumor or a cell in which either the EGFR gene is amplified, or the EGFR protein is overexpressed, or both.

In embodiments, the present disclosure provides a method of treating cancer in a patient having a mutation in at least one gene in the γ-tubulin ring complex, the method comprising administering to the patient an effective amount of a therapy comprising a tubulin polymerization inhibitor. In embodiments, the cancer is glioblastoma. In embodiments, the at least one gene is selected from the group consisting of TUBGCP4 and TUBGCP6. In embodiments, the tubulin polymerization inhibitor comprises an auristatin. In embodiments, the therapy comprising a tubulin polymerization inhibitor is depatuxizumab mafodotin.

In embodiments, the present disclosure provides a method of treating a patient having cancer, the method comprising obtaining a biological sample from the patient, detecting a mutation in a microtubule-related gene in the biological sample, and administering an effective amount of a pharmaceutical composition comprising a tubulin polymerization inhibitor to the patient. In embodiments, the cancer is glioblastoma. In embodiments, the microtubule-related gene is in the γ-tubulin ring complex. In embodiments, the gene in the γ-tubulin ring complex is selected from the group consisting of TUBGCP4 and TUBGCP6. In embodiments, the tubulin polymerization inhibitor comprises an auristatin. In embodiments, the auristatin is MMAE. In embodiments, the auristatin is MMAF. In embodiments, the composition comprises depatuxizumab mafodotin.

In embodiments, the present disclosure provides a method of treating cancer in an individual in need thereof, the method comprising identifying a cancer cell obtained from the individual as having a mutation in the γ-tubulin ring complex, and administering a treatment comprising a tubulin polymerization inhibitor to the patient. In embodiments, the gene in the γ-tubulin ring complex is selected from the group consisting of TUBGCP4 and TUBGP6. In embodiments, the cancer is glioblastoma. In embodiments, the therapy comprising a tubulin polymerization inhibitor comprises an auristatin. In embodiments, the therapy comprising a tubulin polymerization inhibitor is depatuxizumab mafodotin.

In embodiments, the present disclosure provides a method of treating a patient having glioblastoma, where the method comprises obtaining a biological sample from the patient, detecting a mutation in at least one gene in the γ-tubulin ring complex in the biological sample, and, administering a therapy comprising a tubulin polymerization inhibitor to the patient. In embodiments, the at least one gene is selected from the group consisting of TUBGCP4 and TUBGCP6. In embodiments, the therapy comprising a tubulin polymerization inhibitor comprises an auristatin. In embodiments, the therapy comprising a tubulin polymerization inhibitor is depatuxizumab mafodotin.

In embodiments, the present disclosure provides a method for treating glioblastoma, the method comprising administering to a patient in need thereof a therapeutically effective amount of a therapy comprising a tubulin polymerization inhibitor, wherein the patient having glioblastoma has a mutation of a microtubule-related gene. In embodiments, the gene is in the γ-tubulin ring complex. In embodiments, the gene is selected from the group consisting of TUBGCP4 and TUBGCP6. In embodiments, the tubulin polymerization inhibitor is an auristatin. In embodiments, the therapy is depatuxizumab mafodotin.

In embodiments, the present disclosure provides a method of treatment of cancer cells having a mutation in at least one gene in the γ-tubulin ring complex, the method comprising identifying a cancer cell obtained from a human patient having cancer as comprising a mutation in the γ-tubulin ring complex, identifying a treatment comprising a tubulin polymerization inhibitor, and administering to the human patient a therapeutically effective amount of the tubulin polymerization inhibitor. In embodiments, the cancer is glioblastoma. In embodiments, the tubulin polymerization inhibitor is an auristatin. In embodiments, the treatment is depatuxizumab mafodotin.

In embodiments, the present disclosure provides a method for identifying a patient with an increased likelihood of a positive clinical response to a therapy comprising a tubulin polymerization inhibitor, wherein the patient has cancer. In embodiments, the method comprises obtaining a biological sample from the patient, and detecting the presence of a mutation of at least one gene in the γ-tubulin ring complex. In embodiments, the at least one gene is selected from the group consisting of TUBGCP4 and TUBGCP6. Once the patient is identified as having the mutation, the patient can be given a tubulin polymerization inhibitor treatment.

In embodiments of the present disclosure, the cancer is glioblastoma. In embodiments of the present disclosure, the cancer is newly diagnosed glioblastoma. In embodiments of the present disclosure, the cancer is recurrent glioblastoma.

In the embodiments of the present disclosure, the tubulin polymerization inhibitor can be monotherapy, where a tubulin polymerization inhibitor is administered alone, or a combination therapy, where the tubulin polymerization inhibitor is administered with other anti-tumor agents. For example, in embodiments, the tubulin polymerization inhibitor may be administered in combination with radiation and/or temozolomide (TMZ).

In embodiments, the tubulin polymerization inhibitor is an auristatin. In embodiments, the tubulin polymerization inhibitor comprises MMAE. In embodiments, the tubulin polymerization inhibitor comprises MMAF. In embodiments, the therapy comprising a tubulin polymerization inhibitor is depatuxizumab mafodotin.

In embodiments, the present disclosure provides a method of treating cancer in a patient that has both a positive EGFR status (EGFR amplification and/or EGFR overexpression) as well as a mutation in at least one gene in the γ-tubulin ring complex, the method comprising administering to the patient an effective amount of a therapy comprising a tubulin polymerization inhibitor. In embodiments, the cancer is glioblastoma. In embodiments, the at least one gene is selected from the group consisting of TUBGCP4 and TUBGCP6. In embodiments, the tubulin polymerization inhibitor comprises an auristatin. In embodiments, the therapy comprising a tubulin polymerization inhibitor is depatuxizumab mafodotin.

In embodiments, the present disclosure provides a method of treating a patient that has a positive EGFR status cancer (EGFR amplification and/or EGFR overexpression), the method comprising obtaining a biological sample from the patient, detecting a mutation in a microtubule-related gene in the biological sample, and administering an effective amount of a pharmaceutical composition comprising a tubulin polymerization inhibitor to the patient. In embodiments, the cancer is glioblastoma. In embodiments, the microtubule-related gene is in the γ-tubulin ring complex. In embodiments, the gene in the γ-tubulin ring complex is selected from the group consisting of TUBGCP4 and TUBGCP6. In embodiments, the tubulin polymerization inhibitor comprises an auristatin. In embodiments, the auristatin is MMAE. In embodiments, the auristatin is MMAF. In embodiments, the composition comprises depatuxizumab mafodotin.

In embodiments, the present disclosure provides a method of treating a positive EGFR-status cancer (EGFR amplification and/or EGFR overexpression) in an individual in need thereof, the method comprising identifying a cancer cell obtained from the individual as having a mutation in the γ-tubulin ring complex, and administering a treatment comprising a tubulin polymerization inhibitor to the patient. In embodiments, the gene in the γ-tubulin ring complex is selected from the group consisting of TUBGCP4 and TUBGP6. In embodiments, the cancer is glioblastoma. In embodiments, the therapy comprising a tubulin polymerization inhibitor comprises an auristatin. In embodiments, the therapy comprising a tubulin polymerization inhibitor is depatuxizumab mafodotin.

In embodiments, the present disclosure provides a method of treating a patient having glioblastoma which has a positive EGFR status (EGFR amplification and/or EGFR overexpression) in at least one cell, where the method comprises obtaining a biological sample from the patient, detecting a mutation in at least one gene in the γ-tubulin ring complex in the biological sample, and, administering a therapy comprising a tubulin polymerization inhibitor to the patient. In embodiments, the at least one gene is selected from the group consisting of TUBGCP4 and TUBGCP6. In embodiments, the therapy comprising a tubulin polymerization inhibitor comprises an auristatin. In embodiments, the therapy comprising a tubulin polymerization inhibitor is depatuxizumab mafodotin.

In embodiments, the present disclosure provides a method for treating glioblastoma which has a positive EGFR status (EGFR amplification and/or EGFR overexpression) in at least one cell, the method comprising administering to a patient in need thereof a therapeutically effective amount of a therapy comprising a tubulin polymerization inhibitor, wherein the patient having glioblastoma has a mutation of a microtubule-related gene. In embodiments, the gene is in the γ-tubulin ring complex. In embodiments, the gene is selected from the group consisting of TUBGCP4 and TUBGCP6. In embodiments, the tubulin polymerization inhibitor is an auristatin. In embodiments, the therapy is depatuxizumab mafodotin.

In embodiments, the present disclosure provides a method of treatment of cancer cells having both positive EGFR status (EGFR amplification and/or EGFR overexpression) as well as a mutation in at least one gene in the γ-tubulin ring complex, the method comprising identifying a cancer cell obtained from a human patient having cancer as comprising a mutation in the γ-tubulin ring complex, identifying a treatment comprising a tubulin polymerization inhibitor, and administering to the human patient a therapeutically effective amount of the tubulin polymerization inhibitor. In embodiments, the cancer is glioblastoma. In embodiments, the tubulin polymerization inhibitor is an auristatin. In embodiments, the treatment is depatuxizumab mafodotin.

In embodiments, the present disclosure provides a method for identifying a cancer patient with positive EGFR status (EGFR amplification and/or EGFR overexpression) that has an increased likelihood of a positive clinical response to a therapy comprising a tubulin polymerization inhibitor, wherein the patient has cancer. In embodiments, the method comprises obtaining a biological sample from the patient, and detecting the presence of a mutation of at least one gene in the γ-tubulin ring complex. In embodiments, the at least one gene is selected from the group consisting of TUBGCP4 and TUBGCP6. Once the patient is identified as having the mutation, the patient can be given a tubulin polymerization inhibitor treatment.

In embodiments of the present disclosure, the cancer is glioblastoma. In embodiments of the present disclosure, the cancer is newly diagnosed glioblastoma. In embodiments of the present disclosure, the cancer is recurrent glioblastoma.

The following non-limiting examples are illustrative of the present disclosure.

### Examples

Tumor samples for study were from biopsies taken prior to entry onto depatuxizumab mafodotin trial M12-356 (NCT01800695). M12-356 is an open-label, phase 1, 3-arm study: Arm A (depatuxizumab mafodotin+radiation/temozolomide (TMZ) in newly diagnosed GBM; Arm B (depatuxizumab mafodotin+TMZ in newly diagnosed GBM as adjuvant therapy, or in recurrent GBM); and Arm C (depatuxizumab mafodotin monotherapy in recurrent GBM). Arms B and C had and expansion cohort in subjects with centrally confirmed EGFR amplified recurrent GBM.

As discussed in more detail below, whole exome sequencing (WES) was performed on 48 patient tumor samples (44 rGBM and 4 nGBM samples) to identify biomarkers associated with warhead sensitivity. Genes/pathways were identified differentiating responders vs. non-responders and were selected based on highest significance as determined by fold change differentiation, p value, and false discovery rate. siRNA was used to knock down genes of interest in A431 cells (epidermoid carcinoma overexpressing EGFR) to confirm targets. Complete transcriptome sequencing (RNAseq) was performed on 47 patient tumor samples to determine differential gene expression: 43 rGBM samples: responders (R, n=8), non-responders (NR, n=24), and stable disease (SD, n=11).

DNA Isolation and Whole Exome Sequencing

Tumor DNA/RNA was obtained by macrodissecting tumor area (>50% tumor content) from formalin-fixed, paraffin-embedded tumor slides. Matched normal DNA was isolated from lymphocytes of peripheral blood. RNA and DNA was isolated using AIIPrep kit (Qiagen).

Whole exome sequencing was performed on matched tumor/normal DNA samples by Illumina® 2500 or 3000 sequencer (Hayward, Calif.) (2×100 bp or 2×150 bp, respectively) using Agilent SureSelect Clinical Research Exome kits (Cedar Creek, Tex.). Genomic DNA was sonicated to an average size of 175 bp. The fragments were blunt ended, had addition of “A” base to 3′ end, and had Illumina's sequencing adapters ligated to the ends. The ligated fragments underwent amplification for 7 cycles. Fragments were hybridized to biotinylated RNA oligos specific to regions of interest, and selected from remaining fragments using streptavidin beads. Enriched fragments were amplified for 14 cycles with primers that incorporate a unique indexing sequence tag. The resulting libraries were sequenced using the Illumina HiSeq-2500 or HiSeq-3000 as paired end reads extending 101 or 150 bases from both ends of the fragments. Sequencing aimed to achieve a 150× mean on-target coverage for tumor (and 100× for germline DNA).

Whole Exome Sequencing (WES) Data Analysis

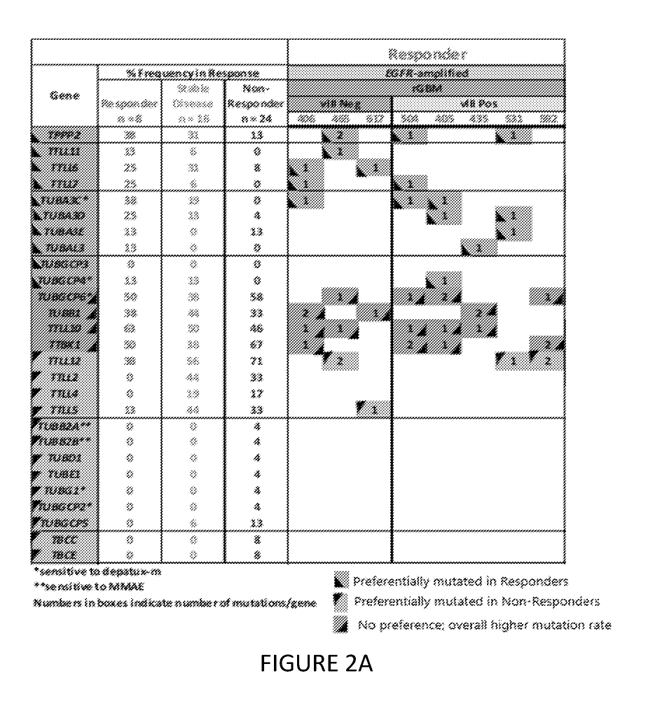

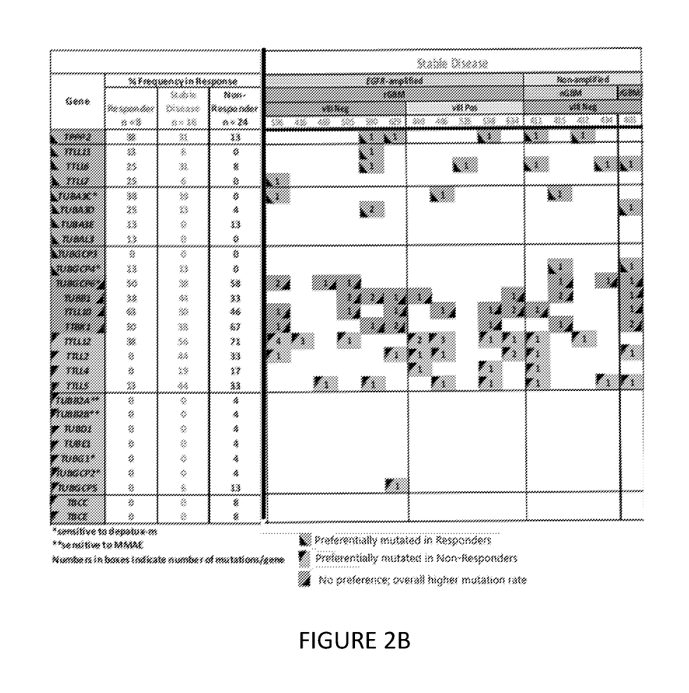

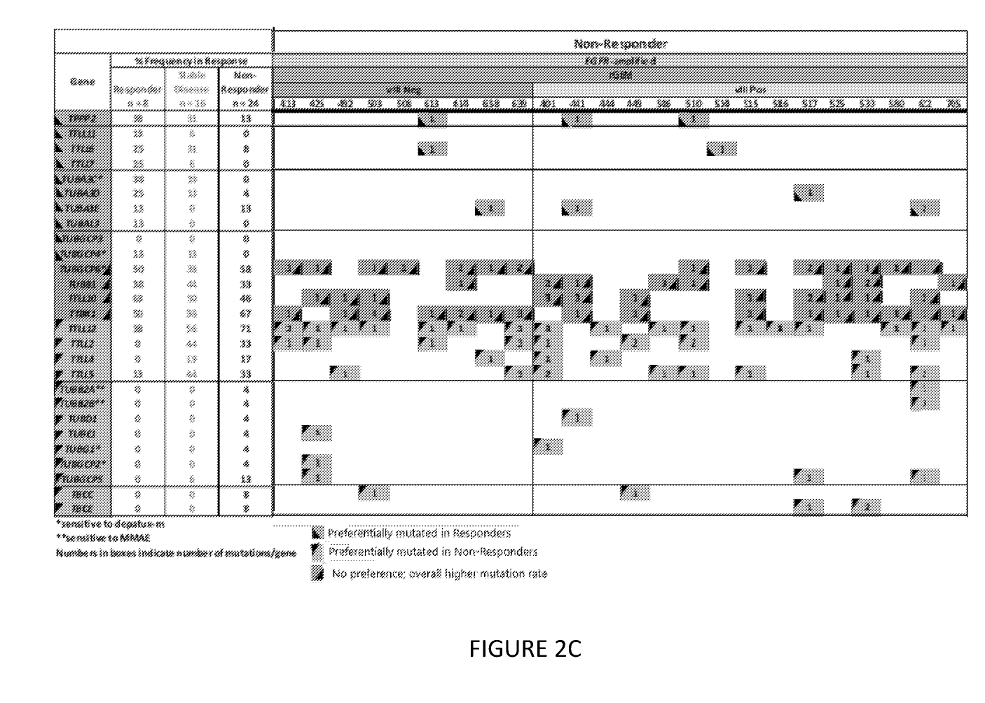

Sequencing results of WES were aligned to human reference genome hg19 using Novoalign (Novocraft Technologies, Petaling Jaya, Malaysia) version (V3.02.06). PCR duplicates were removed from the alignments with Picard and variants were called using VarScan2. Variants were filtered using SnpSift to require a minimal read depth of 10 and variant allele frequency of 0.03 or greater. Common, high frequency SNPS, synonymous, nonframeshift deletions, nonframeshift insertions, NA or unknown variants were removed from downstream analysis. The number of mutations in tubulin-related genes listed in FIG. 2A-C for each patient was compiled and compared to best response as determined by RANO criteria (Wen P Y, Macdonald D R, Reardon D A et-al. Updated response assessment criteria for high-grade gliomas: response assessment in neuro-oncology working group. J. Clin. Oncol. 2010; 28 (11): 1963-72). The tubulin-related genes investigated in FIG. 2A-C are summarized below in Table 1.

TABLE 1
Tubulin Markers

| Gene Symbol | Entrez Gene Name |
|---|---|
| TPPP2 | Tubulin Polymerization Promoting Protein Family |
| | Member 2 |
| TTLL11 | Tubulin Tyrosine Ligase Like 11 |
| TTLL6 | Tubulin Tyrosine Ligase Like 6 |
| TTLL7 | Tubulin Tyrosine Ligase Like 7 |
| TUBA3C | Tubulin Alpha 3c |
| TUBA3D | Tubulin Alpha 3d |
| TUBA3E | Tubulin Alpha 3e |
| TUBAL3 | Tubulin Alpha Like 3 |
| TUBGCP3 | Tubulin Gamma Complex Associated Protein 3 |
| TUBGCP4 | Tubulin Gamma Complex Associated Protein 4 |
| TUBGCP6 | Tubulin Gamma Complex Associated Protein 6 |
| TUBB1 | Tubulin Beta 1 Class VI |
| TTLL10 | Tubulin Tyrosine Ligase Like 10 |
| TTBK1 | Tau Tubulin Kinase 1 |
| TTLL12 | Tubulin Tyrosine Ligase Like 12 |
| TTLL2 | Tubulin Tyrosine Ligase Like 2 |
| TTLL4 | Tubulin Tyrosine Ligase Like 4 |
| TTLL5 | Tubulin Tyrosine Ligase Like 5 |
| TUBB2A | Tubulin Beta 2A Class IIa |
| TUBB2B | Tubulin Beta 2B Class IIb |
| TUBD1 | Tubulin Delta 1 |
| TUBE1 | Tubulin Epsilon 1 |
| TUBG1 | Tubulin Gamma 1 |
| TUBGCP2 | Tubulin Gamma Complex Associated Protein 2 |
| TUBGCP5 | Tubulin Gamma Complex Associated Protein 5 |
| TBCC | Tubulin Folding Cofactor C |
| TBCE | Tubulin Folding Cofactor E |

As shown in FIG. 2A-C, a trend was observed with combinatorial patterns of tubulin genes which were more prevalent in responders vs. non-responders. Some genes in the TUBA, TUBB, TUBG, and TLL gene families were mutated at higher levels across all response groups. Mutations within a single gene did not have significant correlation with best tumor response.

siRNA Experiments

High-Throughput Screen

An siRNA library was produced based targeting genes identified from methods listed above. The siRNA panel in 96 well plates (Dharmacon) was diluted to 66 nM/15 μl was combined with equal volume of RNAiMax Lipofectamine (Life Technologies) diluted to 0.15×/15 μl, pipette mixed thoroughly then allowed to incubate for 15 minutes at room temperature before addition of 30 μl/well to A431, U87MG_del, and U87MG_EGFR cell lines seeded in 96 well plates at 4500 cells/70 μl/well. Plates were incubated in 37° C. 5% CO₂ incubator for 18-24 hours prior to addition of 10 μl drug (MMAE: 1.5 nM final conc., depatuxizumab mafodotin: 18 nM final conc.) or vehicle (phosphate-buffered saline, PBS). Cell viability was assessed post 48 hour drug treatment via Cell Titer Glo (Promega) for A431 cell line or SRB cell stain assay (Sulforhodamine B assay and Chemosensitivity, Voigt et al.) for U87MG_del2.7 and U87MG_EGFR cell lines following recommended protocols with the following exception: media was aspirated off Cell Titer Glo (CTG) plates and 100 μl 1:5 diluted CTG in PBS added per well, covered with foil, rotated to mix 15 minutes prior to reading on Victor chemilluminescent reader. The assay was performed using triplicate plates per drug/vehicle as well as last row of plate contained control siRNA CON3 incubated with vehicle to control for any plate effect. Each plate also contained a row of positive and negative siRNA controls treated with drug for normalization of effects of siRNA KD within and across plates. Effect of siRNA KD assessed via combination of triplicate reactions per siRNA/drug normalized to CON3 siRNA/PBS control and then normalized to CON3 siRNA/drug to determine shift to drug with siRNA KD.

Drug Matrix Screen

A protocol similar to high-throughput screen was used, except siRNA was loaded in a 3×6 well matrix in a 96 well plate with the following siRNAs (CON3, GCNT1, TUBGCP4, TUBGCP6) per quarter plate. EGFR siRNA was loaded in exterior rows as a control. Drug was diluted to 5 serial-5 dilutions (MMAE, Vincristine, Paclitaxel) or 5 serial 3-dilutions (depatuxizumab mafodotin) along with no drug control and added to triplicate wells (starting concentrations MMAE: 100 nM; Vincristine: 2000 nM; Paclitaxel: 2000 nM; depatuxizumab mafodotin: 99 nM). Upon knockdown, A431 cells were more sensitive to depatux-m, MMAE, and vincristine, but this did not increase.

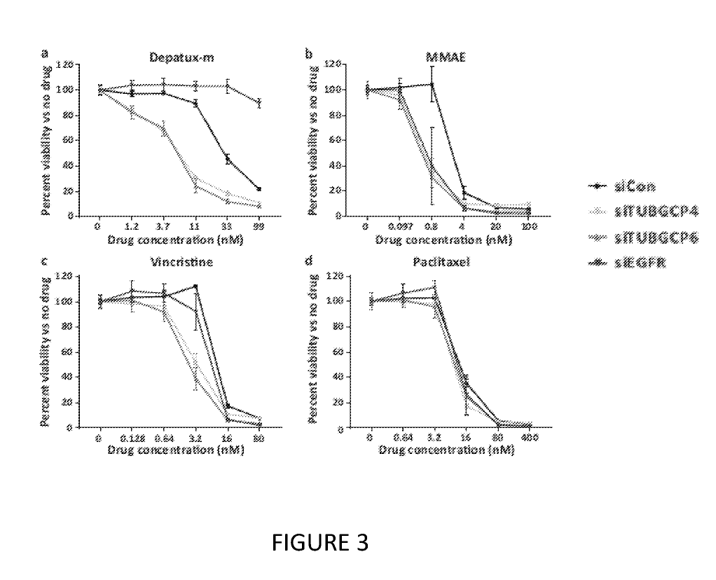

As shown in FIG. 3, results demonstrated a shift in increased sensitivity to all the depolymerizing microtubule agents (MMAE, Vincristine, and depatuxizumab mafodotin) at concentrations of drug yielding <60% cell viability with knockdown of GCNT1, TUBGCP4 and TUBGCP6 in A431 cancer cell line relative to CON3 siRNA control. Specificity to this class of drugs was demonstrated with no additional shift in sensitivity observed with the polymerase microtubule agent, Paclitaxel.

As shown in FIG. 3, siRNA knockdown of differentially mutated genes impacts sensitivity to tubulin polymerization, but not depolymerization, inhibitors. EGFR siRNA served as a control and abrogated drug internalization and cell killing in A431 with depatux-m but not MMAE. Notably, siRNA knockdown of TUBGCP6 and TUBGCP4 demonstrated sensitization to depatux-m in A431 cells. Similar results were achieved when treating cells with other tubulin polymerization inhibitors-monomethyl auristatin E (MMAE) and vincristine—but not the tubulin depolymerization inhibitor paclitaxel, as shown in FIGS. 3b-d.

In addition, knockdown with EGFRsiRNA yielded loss of drug sensitivity only to the EGFR dependent ADC drug, depatuxizumab mafodotin, thus confirming the conditions of the experiment allowed for sufficient gene knockdown.

Depatuxizumab Mafodotin Sensitivity

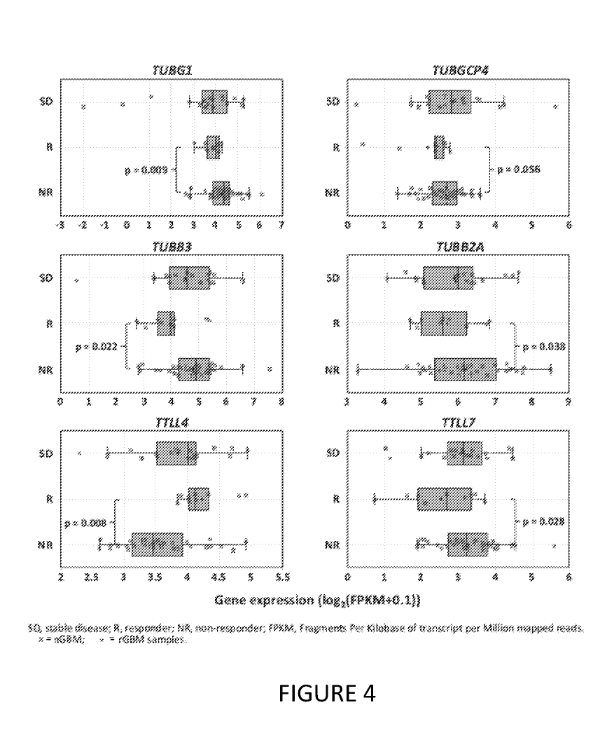

RNAseq performed on rGBM tumor samples resulted in a gene list for differentially expressed genes based on best tumor response to depatux-m. Significant differences in gene expression (p<0.05) of warhead-related genes were observed for TUBG1, TUBB2A, TUBB3, TTLL4, and TTLL7 between responders and non-responders, as shown in FIG. 4.

These examples and embodiments described herein are for illustrative purposes only and various modifications or changes suggested to persons skilled in the art are to the included within the spirit and purview of this application and scope of the appended claims.

What is claimed is:

1. A method of treating cancer in a patient having a mutation in at least one gene in the γ-tubulin ring complex, the method comprising administering to the patient an effective amount of a therapy comprising a tubulin polymerization inhibitor.

2. The method of claim 1, wherein the cancer is glioblastoma.

3. The method of claim 2, wherein the glioblastoma is newly diagnosed glioblastoma.

4. The method of claim 2, wherein the glioblastoma is recurrent glioblastoma.

5. The method of claim 1, wherein the at least one gene is selected from the group consisting of TUBGCP4 and TUBGCP6.

6. The method of claim 1, wherein the tubulin polymerization inhibitor comprises an auristatin.

7. The method of claim 6, wherein the therapy is depatuxizumab mafodotin.

8. A method of treating cancer in an individual in need thereof, the method comprising
identifying a cancer cell from the patient as comprising a mutation in the γ-tubulin ring complex, and
administering a treatment comprising a tubulin polymerization inhibitor to the patient.

9. The method according to claim 8, wherein the cancer is glioblastoma.

10. The method according to claim 9, wherein the glioblastoma is newly diagnosed glioblastoma.

11. The method according to claim 9, wherein the glioblastoma is recurrent glioblastoma.

12. The method according to claim 8, wherein the at least one gene is selected from the group consisting of TUBGCP4 and TUBGCP6.

13. The method according to claim 8, wherein the tubulin polymerization inhibitor comprises an auristatin.

14. The method according to claim 13, wherein the therapy is depatuxizumab mafodotin.

15. A method for treating glioblastoma in an individual in need thereof, wherein the individual has a mutation in a gene in the γ-tubulin ring complex selected from the group consisting of TUBGCP4 and TUBGCP6, the method comprising:
administering to the individual a therapeutically effective amount of depatuxizumab mafodotin.

16. The method of claim 11, wherein the individual has a mutation in a member of the group consisting of: TUBGCP4; TUBGCP6; and both TUBGCP4 and TUBGCP6

17. The method of claim 11, wherein the glioblastoma is newly diagnosed glioblastoma.

18. The method of claim 11, wherein the glioblastoma is recurrent glioblastoma.

19. The method of claim 1, wherein the patient has a positive EGFR status.

20. The method of claim 8, wherein the patient has a positive EGFR status.
